(19) United States
(12) Patent Application Publication
(10) Pub. No.: US 2024/0428907 A1
(43) Pub. Date: Dec. 26, 2024

(54) MULTI-SESSION SUPPORT SYSTEM FOR HEALTHCARE PROFESSIONALS

(71) Applicant: Rowan University, Glassboro, NJ (US)

(72) Inventors: Patrice D. Tremoulet, Moorestown, NJ (US); Andrea Lobo, Glassboro, NJ (US); Christina Simmons, Glassboro, NJ (US); Ganesh Baliga, Glassboro, NJ (US)

(21) Appl. No.: 18/739,516

(22) Filed: Jun. 11, 2024

Related U.S. Application Data
(60) Provisional application No. 63/522,643, filed on Jun. 22, 2023.
(60) Provisional application No. 63/529,829, filed on Jul. 31, 2023.

(51) Int. Cl. G16H 10/60; G16H 40/67

(52) U.S. Cl. CPC G16H 10/60; G16H 40/67

(57) ABSTRACT

A multi-session support system for caregivers that enables quick and easy data collection in a first (notetaking) computing session, synchronously with the delivery of care to multiple patients in a multi-patient care session. Further, the system allows the informal care information notation records developed in the first computing session (e.g., on a mobile phone during a work shift) to be reviewed, edited, augmented, etc. in a second (formal care documentation record creation) computing session, which may be conducted at a later time on a different computing platform (e.g., on a desktop computer after the end of a work shift). Accordingly, the present invention helps to reduce the care documentation burden, by allowing caregivers to take short notes synchronously with the delivery of care, and to prepare formal documentation based on the notes asynchronously with the delivery of care, so that neither patient care nor care documentation are compromised.

### CROSS-REFERENCE TO RELATED APPLICATION

This application claims the benefit of priority, under 35 U.S.C. § 119 (e), of U.S. Provisional Patent Application Nos. 63/522,643, filed Jun. 22, 2023, and 63/529,829, filed Jul. 31, 2023, the entire disclosure of each of which is hereby incorporated herein by reference.

### FIELD OF THE INVENTION

The present invention relates generally to direct support professionals in the field of healthcare/personal care, and more particularly, to a computerized support system for direct support professionals that provides a graphical user interface designed to allow for quick and easy data entry synchronously with the delivery of personal care, in a guided manner designed to encourage complete and accurate documentation of care sessions in accordance with applicable regulatory, insurance reimbursement, corporate and/or other standards.

### DISCUSSION OF RELATED ART

Direct Support Professionals (DSPs) take care of individuals with intellectual and cognitive disabilities who are unable to live independently. The United States is experiencing a DSP crisis, with demand far exceeding supply.

Typically, DSPs (e.g., in the nature of home caregivers) support multiple patients in a single location throughout a work shift, and then at the end of the shift rely upon their memory to start reporting the day's support activities, assistance with feeding, toileting, behavioral and medication aspects of caregiving, etc. (e.g., using paper-based documents, a home PC in a home office, employer's office, etc.). Accordingly, the recordkeeping and care documentation are performed asynchronously with the delivery of care, largely due to a prioritization that prioritizes delivery of care to people in need over robust documentation of such care.

Although generating care documentation is a critical responsibility, it is one of the least rewarding aspects of DSPs' jobs. Additionally, it can be challenging to create records, and/or create accurate records, asynchronously.

Technology solutions might help boost effectiveness and increase job satisfaction, which in turn could improve the quality of life of individuals they support. However, DSPs, with varied ages, levels of education, and comfort interacting with technology, are not likely to adopt tools that detract from caregiving responsibilities or increase workload, so technology for them must be relatively simple, extremely intuitive, and provide highly-valued capabilities.

Further, existing software tools have been designed primarily for staff who provide one-on-one support for children with disabilities, and thus lack patient management functional required for Caregivers who simultaneously care for multiple persons.

Organizations that employ DSPs would be interested in purchasing effective systems/tools to help them attract and retain workers and to help improve the quantity and quality of data that DSPs produce and share.

Moreover, organizations such as home caregivers and nursing homes, are also facing acute staffing shortages since many left this field during and subsequent to the Covid-19 pandemic. Those organizations would also benefit from, and be willing to invest in, commercial technologies that provide the necessary functionality to their workforces.

What is needed is a support system for caregivers that enables and facilitates data collection in a quick and easy fashion, such that it can be performed synchronously with the delivery of care, and that supports robust healthcare record creation at the end of a caregiving work shift.

### SUMMARY

The present invention provides a multi-session support system for caregivers that enables and facilitates data collection in a quick, easy and straightforward fashion in a first (notetaking) computing session, such that it can be performed synchronously with the delivery of care, and that supports robust healthcare record creation based on the notes in a second (formal care documentation record creation) computing session.

Accordingly, the present invention provides a support system providing a graphical user interface that is very simple and easy to use for caregivers, and thus suitable for use to gather/record relevant data synchronously with the delivery of care (e.g., during delivery of care, on a patient-by-patient basis), and informally and easily in a first computing session, e.g. on a first (e.g., mobile) computing platform. Accordingly, the present invention helps to reduce documentation burden, allowing Caregivers to use the system in accordance with patient care and documentation workflows, to avoid the high burden of interacting with a sophisticated system, and to spend more time and energy on more gratifying aspects of their jobs, all while logging all pertinent patient and care details, etc.

This in turn tends to increase job satisfaction, increase effectiveness, and decrease turnover among caregivers, particularly DSPs, for whom there is a severe shortage in the workforce.

More particularly, the present invention enables the caregiver to use a smartphone to write/record personal notes (informal care information notation records) all shift long, while caring for multiple patients concurrently, e.g., in real time (synchronously with the delivery of care), to keep contemporaneous records, in a first (notetaking) computing session.

Still further, the support system is configured to import the informal care information notation records gathered via the smartphone into a web-based platform with which the caregiver can interact asynchronously with the delivery of care to review, augment or otherwise transfer the synchronously-created notes into robust care documentation compliant with regulatory, corporate, or industry standards in a second computing session, which may be on a second (e.g., web) computing platform. For example, the present invention provides a computerized (e.g., web-based) user interface that allows the caregiver to, e.g., at the end of the shift, login to a web-based platform to which the notes have been imported from the smartphone, and use the web-based platform to categorize those notes and formalize those notes such that they can be incorporated into robust care documentation records suitable for inclusion in an EMR/EHR system.

Accordingly, the present invention provides a multi-session support system that: i) enables quick, easy in-the-moment data collection; and ii) allows Caregivers to access a private secure web portal to review, sort, filter, and organize their notes to facilitate end of shift documentation. An exemplary support system includes four components: mobile device/smartphone “app” software that runs on conventional smartphone, etc. hardware to provide a graphical user interface in accordance with the present invention; a private, secure web “app”/website/software platform that imports data gathered via the mobile app and provides a graphical user interface in accordance with the present invention that allows Caregivers to access, review, and organize notes created with via the mobile app interface; a cloud-based data center that houses the relevant data; and an administrative website/software platform for creating and managing user accounts.

### BRIEF DESCRIPTION OF THE FIGURES

An understanding of the following description will be facilitated by reference to the attached drawings, in which:

FIG. 1 is a system diagram showing an exemplary network computing environment in which a Multi-Session Support System for caregivers in accordance with the present invention may be employed;

FIG. 2 is a schematic block diagram illustrating an exemplary embodiment of a computerized Caregiver Notation Device of FIG. 1 in accordance with an exemplary embodiment of the present invention;

FIG. 3 is a schematic block diagram illustrating an exemplary embodiment of a computerized Multi-Session Support System of FIG. 1 in accordance with an exemplary embodiment of the present invention;

FIG. 4 is a flow diagram illustrating a method of operation of an exemplary Caregiver Notation Device to create informal care documentation notes in accordance with an exemplary embodiment of the present invention; and

FIG. 5 is a flow diagram illustrating a method of operation of an exemplary Multi-Session Support System to create formal care documentation records in accordance with an exemplary embodiment of the present invention;

FIG. 6 shows an exemplary graphical user interface window displayed by an exemplary Caregiver Notation Device in accordance with an exemplary embodiment of the present invention; and

FIG. 7 shows an exemplary graphical user interface window displayed by an exemplary Multi-Session Support System in accordance with an exemplary embodiment of the present invention.

### DETAILED DESCRIPTION

The present invention provides a multi-session support system for caregivers that enables and facilitates data collection in an easy and straightforward fashion, such that it can be performed synchronously with the delivery of care, and that supports robust healthcare record creation. The present invention provides a support system providing a graphical user interface that is very simple and easy to use for Caregivers, and thus suitable for use to gather/record relevant data synchronously with the delivery of care (e.g., during delivery of care, on a patient-by-patient basis), and informally and easily in a first computing session, e.g. on a first (e.g., mobile) computing platform. Accordingly, the present invention helps to reduce documentation burden, allowing Caregivers to use the system in accordance with patient care and documentation workflows, to avoid the high burden of interacting with a sophisticated system, and to spend more time and energy on more gratifying aspects of their jobs, all while logging all pertinent patient and care details, etc.

This in turn tends to increase job satisfaction, increase effectiveness, and decrease turnover among Caregivers, particularly DSPs, for whom there is a severe shortage in the workforce.

More particularly, the present invention enables the caregiver to use a smartphone to write/record personal notes (informal care information notation records) all shift long, while caring for multiple patients concurrently, e.g., in real time (synchronously with the delivery of care), to keep contemporaneous records, in a first (notetaking) computing session.

Still further, the support system is configured to import the informal care information notation records gathered via the smartphone into a web-based platform with which the caregiver can interact asynchronously with the delivery of care to review, augment or otherwise transfer the synchronously-created notes into robust care documentation compliant with regulatory, corporate, or industry standards in a second computing session, which may be on a second (e.g., web) computing platform. For example, the present invention provides a computerized (e.g., web-based) user interface that allows the caregiver to, e.g., at the end of the shift, login to a web-based platform to which the notes have been imported from the smartphone, and use the web-based platform to categorize those notes and formalize those notes such that they can be incorporated into robust care documentation records suitable for inclusion in an EMR/EHR system.

Accordingly, the present invention provides a multi-session support system that: i) enables quick, easy in-the-moment data collection; and ii) allows Caregivers to access a private secure web portal to review, sort, filter, and organize their notes to facilitate end of shift documentation. An exemplary support system includes four components: mobile device/smartphone “app” software that runs on conventional smartphone, etc. hardware to provide a graphical user interface in accordance with the present invention; a private, secure web “app”/website/software platform that imports data gathered via the mobile app and provides a graphical user interface in accordance with the present invention that allows Caregivers to access, review, and organize notes created with via the mobile app interface; a cloud-based data center that houses the relevant data; and an administrative website/software platform for creating and managing user accounts.

Accordingly, the multi-session support system both reduces the burden associated with producing care documentation and increases the quality (level of detail and accuracy) of that documentation, and further is capable of managing a one-to-many or a few-to-many caregiver-to-patient dynamic, in contrast to many existing tools that have been designed primarily for staff who provide one-to-one care for persons with disabilities.

The following detailed description of an exemplary embodiment of the present invention contains many specifics for the purpose of illustration. Anyone of ordinary skill in the art will appreciate that many variations and alterations to the following details are within the scope of the present invention. Accordingly, the following implementations of the present invention are set forth without any loss of generality to, and without imposing limitations upon, the claimed invention.

### System Environment

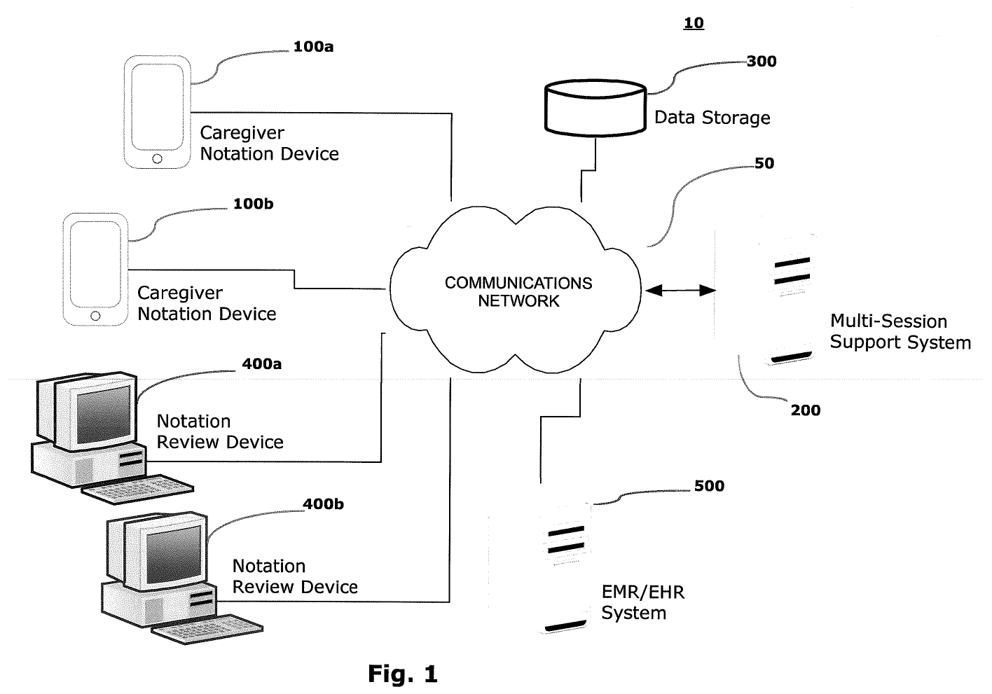

An exemplary embodiment of the present invention is discussed below for illustrative purposes. FIG. 1 is a system diagram showing an exemplary network computing environment 10 in which the present invention may be employed. As shown in FIG. 1, the exemplary network environment 10 includes certain conventional computing hardware and software for communicating via a communications network 50, such as the Internet, etc., using a Caregiver Notation Device (CND) 100 (e.g., devices 100a and 100b) each of which may be, for example, one or more personal computers/PCs, laptop computers, tablet computers, smartphones, smartwatches, or other computing system hardware, including computerized/networked communication hardware/software/functionality, such as computer-based and/or cloud-based servers, and the like, or other so-called “connected” communication devices having communication capabilities for communicating data via the network and/or locally to other devices.

Additionally, as shown in FIG. 1, the exemplary network environment 10 includes a Multi-Session Support System (MSSS) 200 including certain conventional computing hardware and software for communicating via a communications network 50, such as the Internet, etc., which include, for example, as a web-server or other cloud-based computing system hardware, including computerized/networked communication hardware/software/functionality, such as computer-based and/or cloud-based servers, and the like, or other so-called “connected” communication devices having communication capabilities for communicating data via the network and/or locally to other devices.

The CNDs 100a, 100b and the MSSS 200 are provided and specially-configured in accordance with the present invention as described here. The exemplary network environment 10 further includes cloud-based data storage 300, an Electronic Medical Records/Electronic Health Records system 500, and a Network-Based Notes Review Device 400 (such as a personal computer, laptop computer, or tablet computer 400a, 400b), each of which may be conventional systems These devices need not be specially configured with software/functionality in accordance with the present invention, but may be configured to communicate via the communications network 50 using conventional hardware and software for communication with other devices in the network 10 in accordance with the teachings of the present invention.

In accordance with a certain aspect of the present invention, one or more of the CNDs 100a, 100b is a smartphone or other computing device configured to store and execute an “app” or other purpose-specific application software in accordance with the present invention, although this is not required in all embodiments.

### Caregiver Notation Device

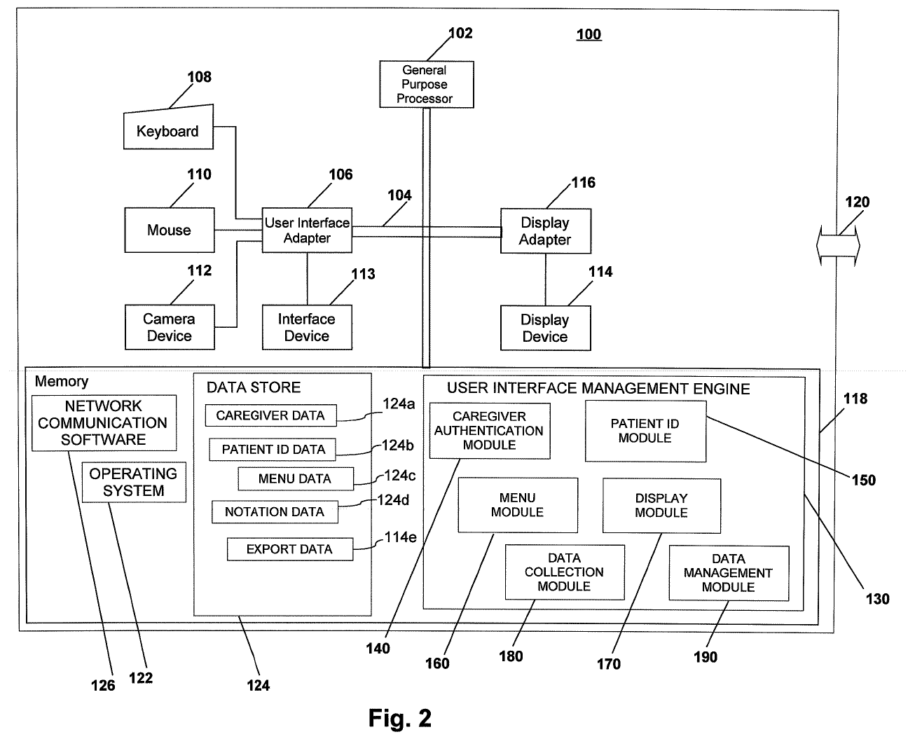

FIG. 2 is a schematic block diagram showing an exemplary Caregiver Notation Device (CND) 100 (e.g., 100a or 100b) in accordance with an exemplary embodiment of the present invention. The exemplary CND 100 is a special-purpose computerized system that includes conventional computing hardware storing and executing both conventional software enabling operation of a general-purpose computing system, such as operating system software, network communications software, and specially-configured computer software for configuring the general-purpose hardware as a special-purpose computer system for carrying out at least one method in accordance with the present invention. By way of example, the communications software may include conventional web server software, and the operating system software may include IOS, Android, Windows, Linux software.

Referring again to FIG. 2, there is illustrated a block diagram of an exemplary CND 100 according to some embodiments is shown. In some embodiments, the CND 100 may, for example, execute, process, facilitate, and/or otherwise be associated with some or all of the functionality of the embodiments described herein.

Accordingly, the exemplary CND 100 of FIG. 2 includes a general-purpose processor, such as a microprocessor (CPU) 102 and a bus 104 employed to connect and enable communication between the processor 102 and the components of the CND system in accordance with known techniques. In some embodiments, the processor 102 may be or include any type, quantity, and/or configuration of processor that is or becomes known. In some embodiments, the processor 102 may comprise multiple inter-connected processors, microprocessors, and/or micro-engines. According to some embodiments, the processor 102 (and/or the CND system 100 and/or other components thereof) may be supplied power via a power supply (not shown), such as a battery, an Alternating Current (AC) source, a Direct Current (DC) source, an AC/DC adapter, solar cells, and/or an inertial generator.

The exemplary CND 100 includes a user interface adapter 106, which connects the processor 102 via the bus 104 to one or more interface devices, such as a keyboard 108, mouse 110, and/or other interface devices 112, which can be any user interface device, such as a touch-sensitive screen, digitized entry pad, etc. The bus 104 also connects a display device 114, such as an LCD screen or monitor, to the processor 102 via a display adapter 116.

The bus 104 also connects the processor 102 to memory 118, which can include a solid-state drive, an optical drive, a diskette drive, a tape drive, etc., or any other appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 118 may, according to some embodiments, store one or more software components. Any or all of the exemplary instructions and data types described herein and other practicable types of data may be stored in any number, type, and/or configuration of memory systems that is or becomes known. The memory 118 may, for example, comprise one or more data tables or files, databases, table spaces, registers, and/or other storage structures. In some embodiments, multiple databases and/or storage structures (and/or multiple memory systems) may be utilized to store information associated with the system 100. According to some embodiments, the memory 118 may be incorporated into and/or otherwise coupled to the system 100 (e.g., as shown) or may simply be accessible to the system 100 (e.g., externally located and/or situated).

The CND 100 may communicate with other computers or networks of computers, for example via a communications channel, network card, modem, transmitter, receiver, or transceiver (collectively, “transceiver”) 120. In some embodiments, the transceiver 120 may comprise any type or configuration of communication system that is or becomes known or practicable. The transceiver 120 may, for example, comprise a Network Interface Card (NIC), a telephonic system, a cellular or other mobile computing network system, a router, a hub, a modem, and/or a communications port or cable. According to some embodiments, the transceiver 120 may also or alternatively be coupled to the processor 102. In some embodiments, the transceiver 120 may comprise an IR, RF, Bluetooth™, Near-Field Communication (NFC), and/or Wi-Fi® network system coupled to facilitate communications between the processor 102 and another system (not shown). The CND 100 may be associated with such other computers in a local area network (LAN) or a wide area network (WAN), and may operate as a server in a client/server arrangement with another computer, etc. Such configurations, as well as the appropriate communications hardware and software, are known in the art.

The CND 100 is specially configured in accordance with the present invention. Accordingly, as shown in FIG. 2, the CND 100 includes computer-readable, processor-executable instructions stored in the memory 118 for carrying out the methods described herein. Further, the memory 118 stores certain data, e.g., in one or more databases or other data stores 124 shown logically in FIG. 2 for illustrative purposes, without regard to any particular embodiment in one or more hardware or software components.

Further, as will be noted from FIG. 2, the CND 100 includes, in accordance with the present invention, a User Interface Management Engine (UIME) 130, shown schematically as stored in the memory 118, which includes a number of additional modules providing functionality in accordance with the present invention, as discussed in greater detail below. These modules may be implemented primarily by specially-configured software including microprocessor-executable instructions stored in the memory 118 of the CND 100. Optionally, other software may be stored in the memory 118 and and/or other data may be stored in the data store 124 or memory 118. Further, the UIME 130 includes one or more modules shown logically in FIG. 2 for illustrative purposes, without regard to any particular embodiment in one or more hardware or software components.

It should be noted that some of the wording and form of description herein is done to meet applicable statutory requirements. Although the terms “step”, “block”, “module”, “engine”, etc. might be used herein to connote different logical components of methods or systems employed and/or for ease of illustration, the terms should not be interpreted as implying any particular order among or between various steps herein disclosed unless and except when the order of individual steps is explicitly described, or be interpreted as implying any distinct structure separate and apart from other structures of the system.

As shown in FIG. 2, the CND 100 includes a data store 124 and a UIME 130 in accordance with the present invention.

In part, the CND 100 stores Caregiver Data 124a in the data store 124. The Caregiver Data 124a identifies the caregiver/operator of the CND 100 and includes any relevant caregiver-identifying and caregiver-associated data, such as caregiver name, employee number, license number, employee identification code, contact information and any other information identifying the caregiver, the caregiver's employer and/or any other associated information suitable for inclusion in a medical record or healthcare record (e.g., in an EMR/EHR electronic records system) and/or relevant to processing of an associated insurance or other claim. By way of example, some or all of this information may be provided by or gathered from the user by direct input via the keyboard 108, mouse 110, camera 112, touchscreen or other interface device 113 of the CND 100, or by data communication via the network 50 with another computing device.

Further, the CND 100 stores Patient Data 124b in the data store 124. The Patient Data 124b identifies the patent and includes any relevant patient-identifying and/or patient-associated data, such as patient name and other information identifying the patient and/or an associated medical record, e.g., of an EMR/EHR electronic records system. By way of example, some or all of this information may be provided by or gathered from the caregiver (or patient) by direct input via the keyboard 108, mouse 110, camera 112, touchscreen or other interface device 113 of the CND 100, or by data communication via the network 50 with another computing device.

Still further, the exemplary CND 100 stores data for creating and display menus as Menu Data 124c in the data store 124. The Menu Data 124c may be gathered via the CND 100 as user input via input devices, or may be generated (by the Menu Module 160) as a function of stored data (e.g., Patient Data 124b), e.g., as a list of patient names/identifier codes in a Patient Menu, and/or other stored data, e.g., a list of daily activity types to be displayed in an Activity Menu.

Further still, the exemplary CND 100 stores collected notation data as Collected Data 124d in the data store 124. The Collected Data 124d is data gathered from the caregiver via the CND 100. For example, such data may be gathered via a microphone interface or touchscreen interface device 113, a keyboard device 108, etc. to gather the caregiver's notation (voice recordings, text input, menu selections, etc.) that are effectively notes that serve as shorthand notation that can be used to document care given by the caregiver to a patient. For example, the Collected Data 124d may include data collected by the CND 100 as data input provided by the caregiver to a particular patient synchronously with the caregiver's care delivery session delivery care to the particular patient.

Additionally, the exemplary CND 100 stores data for exporting of data to a remotely-located, e.g., “in the cloud”, Multi-Session Support System (MSSS) 200, in accordance with the present invention. Such data is stored as Export Data 124e in the data store 124. The Export Data 124e may be data provide as a result of a pre-configuration or setup process, or provided as user input at the CND 100 (e.g., under control of the Data Management Module) that provides information usable to transmit/synch the data to the MSSS, a remote data store 300 (e.g., in the cloud). A remove 9 e.g., in the cloud) EMR/EHR system 500, etc., and/or may include information about which data to export (e.g., Patient Data, Collected Data, Caregiver Data, etc.) and/or provide additional information to be included as part of any export (e.g., information that may be generic to patients but includable for every export of data for ap articular caregiver, caregiver employer, etc.).

The exemplary User Interface Management Engine (UIME) 130 includes a Caregiver Authentication Module (CAM) 140 that is operable to manage display of appropriate graphical user interface windows via the display device 114 of the CND 100, and to authenticate a caregiver as an authorized system user (e.g., via communication with the MSSS 200 and/or checking of previously-stored Caregiver Data 124a), gather relevant caregiver-identifying and/or associated data and store such data as the Caregiver Data 124a in the data store 124. If a user is authenticated by the CAM 140, then the UIME 130 provides the user access to the functionality of the UIME 130 for the purposes described herein.

The exemplary UIME 130 also includes a Patient ID Module (PIDM) 150 that is operable to manage display of appropriate graphical user interface windows via the display device 114 of the CND 100, and to create a patient record by gathering and storing patient-identifying and/or patient-associated data and store such data as the Patent ID Data 124b in the data store 124. In one embodiment, the PIDM 150 (in cooperation with a Display Module 170) may provide a graphical user interface allowing a caregiver to create/add a record for a new patient identifier (ID) associated with a patient, such as the first 2 letters of the first name and the first 2 letters of the last name. If no client is identified, the note may at least initially be classified in a generic “all shift long” category.

After a patient record is created by the PIDM 150, then the patient/patient's record will be displayed by the Menu Module 160 to provide a caregiver-selectable option in an appropriate graphical user interface window displayed on the display device so that the caregiver can select the patient and record associated notations as part of a care record, etc.

The exemplary UIME 130 also includes a Menu Module (MM) 160 that is operable to manage display of appropriate graphical user interface windows via the display device 114 of the CND 100. More particularly, the MM 160 is responsible for determining which user-selectable menu options should be displayed in each menu, e.g., as a function of predetermined settings/data, user input, or a recent analysis of stored data, such as stored Patient ID Data 124b and/or Menu Data 124c. For example, the MM 160 may manage display of user selectable options that may include one or more of a Patient ID Menu, an Activity Menu, a Recorded Note Menu (button), and a Textual Note Menu (text entry field) (in additional to a keyboard and/or other buttons as needed). The options are user-selectable by the caregiver to aid in quick and easy creation of patient care documentation notations/notes in a quick and easy fashion sufficient to permit creation of such notations on a patient-by-patient basis (unique to each patient) synchronously with the delivery of care (e.g., as part of each multi-patient care session).

The exemplary UIME 130 further includes a Display Module (DM) 170 that is operable to cause display of appropriate graphical user interface windows and menus/user-selectable menu options via the display device 114 of the CND 100, working in concert with the CAM 140, PIDM 150, and MM 160, to provide the graphical user interfaces and functionality of the CND 100 for the purposes described herein.

The exemplary UIME 130 also includes a Data Collection Module (DCM) 180 that is operable to receive user-input data via the user interface windows and/or interface devices 113 of the CND 100. The DCM 180 works in concert with the CAM 140, PIDM 150, and MM 160, to cause storage of the Caregiver Data 124a, Patient ID Data 124b, and Menu Data 124c. Additionally, the DCM 180 works to receive data via the user interface windows and/or interface devices 113 of the CND 100 that make up the caregiver's notes for care to patients in a care session (e.g., the caregiver's input of a patient ID, a selected Activity, textual input and/or a recording associated with the Patient ID and/or selected Activity, etc., and stores that data as Collected Data 124d in the data store 124. This information is effectively the caregiver's notation/notes, which can be recorded synchronously with a patient care session for multiple patients. Additionally, the DCM 180 may add data to a notation record, or each notation record, as desired. For example, the DCM 180 may autonomously add a timestamp (e.g., date and time) indicating the time of creation of each record and/or input (e.g., by gathering such data from operating system software of the CND 100). In this way, the data input by the caregiver is autonomously augmented to make a more robust care record, e.g., to comply with industry, regulatory and/or other requirements.

Further, the exemplary UIME 130 also includes a Data Management Module (DMM) 190 that is operable to transmit, synch, and/or export patient notation data gathered at the CND 100 to the remote data storage 300/MSSS 200 for subsequent review, verification, completion, augmentation into a final/formal patient care record. The DMM 190 may retrieve stored Export Data 124e, which may indicate how gathered data should be handled, so that the DMM can take appropriate action accordingly. For example, the Export Data 124e may provide network address information for transmitting data to the MSSS 200, and/or to a data store 300. By way of further example, the Export Data 124e may indicate which data should be transmitted from the CND 100, and/or add additional information to the notation record, e.g. caregiver-identifying/associated data retrieved from the Caregiver Data 124a, patient identifying/associated data gathered from the Patient ID Data 124b, etc. In this way, the DMM 190 exports/transmits data to the MSSS 200 necessary to permit the caregiver to review the notations and/or create a complete/robust care record, e.g., to comply with industry, regulatory and/or other requirements.

### Multi-Session Support System

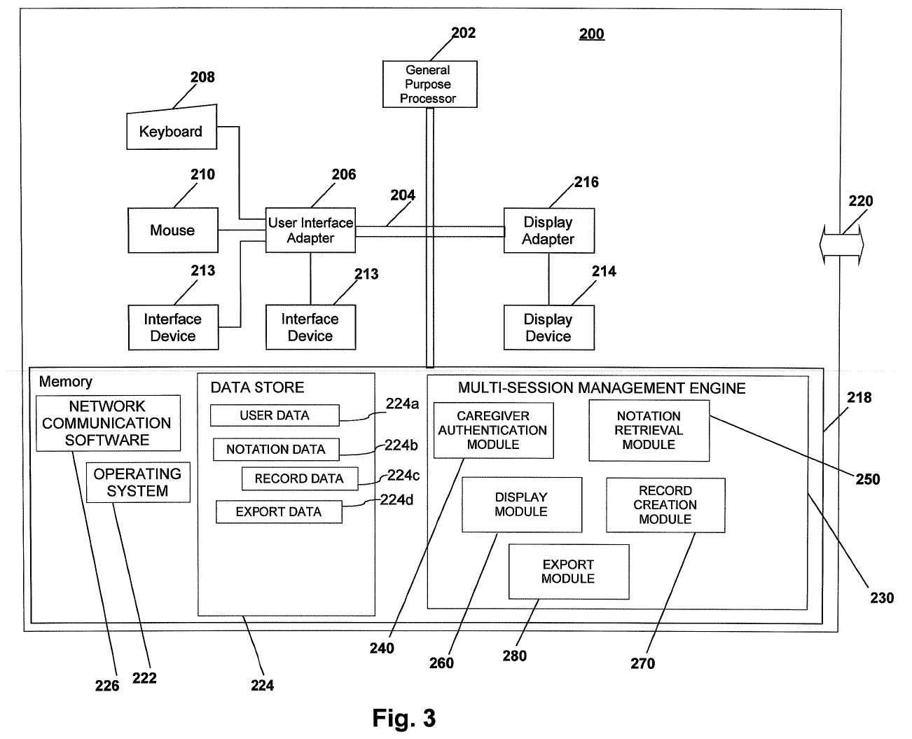

FIG. 3 is a schematic block diagram showing an exemplary Multi-Session Support System (MSSS) 200 in accordance with an exemplary embodiment of the present invention. The exemplary MSSS 200 is a special-purpose computerized system that includes conventional computing hardware storing and executing both conventional software enabling operation of a general-purpose computing system, such as operating system software, network communications software, and specially-configured computer software for configuring the general-purpose hardware as a special-purpose computer system for carrying out at least one method in accordance with the present invention. By way of example, the communications software may include conventional web server software, and the operating system software may include iOS, Android, Windows, Linux software.

Accordingly, the exemplary MSSS 200 of FIG. 3 includes a general-purpose processor, such as a microprocessor (CPU), 202 and a bus 204 employed to connect and enable communication between the processor 202 and the components of the MSSS system in accordance with known techniques. In some embodiments, the processor 202 may be or include any type, quantity, and/or configuration of processor that is or becomes known. In some embodiments, the processor 202 may comprise multiple inter-connected processors, microprocessors, and/or micro-engines. According to some embodiments, the processor 202 (and/or the system 200 and/or other components thereof) may be supplied power via a power supply (not shown), such as a battery, an Alternating Current (AC) source, a Direct Current (DC) source, an AC/DC adapter, solar cells, and/or an inertial generator. In the case that the system 200 comprises a server, such as a blade server, necessary power may be supplied via a standard AC outlet, power strip, surge protector, and/or Uninterruptible Power Supply (UPS) system.

The exemplary MSSS 200 includes a user interface adapter 206, which connects the processor 202 via the bus 204 to one or more interface devices, such as a keyboard 208, mouse 210, and/or other interface devices 213, which can be any user interface device, such as a touch-sensitive screen, digitized entry pad, etc. The bus 204 also connects a display device 214, such as an LCD screen or monitor, to the processor 202 via a display adapter 216.

The bus 204 also connects the processor 202 to memory 218, which can include a hard drive, a solid-state drive, an optical drive, a diskette drive, a tape drive, etc. The memory 218 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 218 may, according to some embodiments, store one or more software components. Any or all of the exemplary instructions and data types described herein and other practicable types of data may be stored in any number, type, and/or configuration of memory systems that is or becomes known. The memory 218 may, for example, comprise one or more data tables or files, databases, table spaces, registers, and/or other storage structures. In some embodiments, multiple databases and/or storage structures (and/or multiple memory systems) may be utilized to store information associated with the system 200. According to some embodiments, the memory 218 may be incorporated into and/or otherwise coupled to the system 200 (e.g., as shown) or may simply be accessible to the system 200 (e.g., externally located and/or situated).

The MSSS 200 may communicate with other computers or networks of computers, for example via a communications channel, network card, modem, transmitter, receiving, or transceiver (collectively, “transceiver”) 220. In some embodiments, the transceiver 220 may comprise any type or configuration of communication system that is or becomes known or practicable. The transceiver 220 may, for example, comprise a Network Interface Card (NIC), a telephonic system, a cellular network system, a router, a hub, a modem, and/or a communications port or cable. According to some embodiments, the transceiver 220 may also or alternatively be coupled to the processor 202. In some embodiments, the transceiver 220 may comprise an IR, RF, Bluetooth™, Near-Field Communication (NFC), and/or Wi-Fi® network system coupled to facilitate communications between the processor 202 and another system (not shown). The MSSS 200 may be associated with such other computers in a local area network (LAN) or a wide area network (WAN), and may operate as a server in a client/server arrangement with another computer, etc. Such configurations, as well as the appropriate communications hardware and software, are known in the art.

The MSSS 200 is specially configured in accordance with the present invention. Accordingly, as shown in FIG. 3, the MSSS 200 includes computer-readable, processor-executable instructions stored in the memory 218 for carrying out the methods described herein. Further, the memory 218 stores certain data, e.g., in one or more databases or other data stores 224 shown logically in FIG. 3 for illustrative purposes, without regard to any particular embodiment in one or more hardware or software components.

Further, as will be noted from FIG. 3, the MSSS 200 includes, in accordance with the present invention, a Multi-Session Management Engine (MPME) 230, shown schematically as stored in the memory 218, which includes a number of additional modules providing functionality in accordance with the present invention, as discussed in greater detail below. These modules may be implemented primarily by specially-configured software including microprocessor-executable instructions stored in the memory 218 of the MSSS 200. Optionally, other software may be stored in the memory 218 and and/or other data may be stored in the data store 224 or memory 218. Further, the MPME 230 includes one or more modules shown logically in FIG. 3 for illustrative purposes, without regard to any particular embodiment in one or more hardware or software components.

It should be noted that some of the wording and form of description herein is done to meet applicable statutory requirements. Although the terms “step”, “block”, “module”, “engine”, etc. might be used herein to connote different logical components of methods or systems employed and/or for ease of illustration, the terms should not be interpreted as implying any particular order among or between various steps herein disclosed unless and except when the order of individual steps is explicitly described, or be interpreted as implying any distinct structure separate and apart from other structures of the system.

As shown in FIG. 3, the MSSS 200 includes a data store 224 and a Multi-Session Management Engine (MPME) 230 in accordance with the present invention.

The MSSS 200 stores Caregiver Data 224a in the data store 224. The Caregiver Data 224a identifies authorized caregivers and includes any relevant caregiver-identifying and caregiver-associated data, such as caregiver name, employee number, license number, employee identification code, contact information and any other information identifying the caregiver, the caregiver's employer and/or any other associated information suitable for inclusion in a medical record or healthcare record (e.g., in an EMR/EHR electronic records system) and/or relevant to processing of an associated insurance or other claim. By way of example, some or all of this information may be provided by or gathered from the user by direct input via an interface device 113 of a CND 100, or by data communication via the network 50 with another computing device.

Further, the exemplary MSSS 200 stores Notation Data 224b in the data store 124. The Notation Data 224b includes notation records created by one or more caregivers at one or more CNDs 100, as well as any other additional information as exported from the CNDs 100. In certain embodiments, this Notation Data 224b may be transmitted/exported directly from the CNDs 100 to the MSSS 200. In other embodiments, caregiver-created notation records may be exported from various CNDs 100 and stored in cloud-based storage 300, and then be retrieved as needed by the MSSS 200, and optionally stored as Notation Data 224b in the data store 224 of the MSSS 200.

Still further, the exemplary MSSS 200 stores patient record data as Record Data 224c. The patient records data capturing the final/definitive patient care documentation that started as caregiver notations recorded at the CND 100, and that was then reviewed/verified/corrected/augmented/completed after subsequent review via a second computing session (e.g., via a mobile, online, web, or personal computer computing platform) different from the mobile computing session of the CND 100, in accordance with the present invention, as discussed further below.

Further still, the exemplary MSSS 100 stores data providing information for exporting of the Record Data 224d for each patient to one or more remotely-located, e.g., “in the cloud”, systems, such as cloud-based data storage 300 and/or one or more EMR/EHR systems 500, as appropriate for each respective patient associated with each record. The Export Data 224d may be data provided as a result of a pre-configuration or setup process, or provided as user input at the MSSS 200 (e.g., under control of the Record Creation Module) that provides information usable to transmit/synch the data to the remote data store 300 and/or EMR/EHR system 500, etc., and/or may include information about which Record Data 224c to export for each patient and/or provide additional information to be included as part of any export.

The exemplary Multi-Session Management Engine (MPME) 230 includes a Caregiver Authentication Module (CAM) 240 that is operable to manage display of appropriate graphical user interface windows via the display device 114 of the CND 100, and to authenticate a caregiver as an authorized system user (e.g., via communication with the CND 100 and/or checking of previously-stored Caregiver Data 224a), gather relevant caregiver-identifying and/or associated data and store such data as the Caregiver Data 224a in the data store 224. If a user is authenticated by the CAM 140, then the MPME 130 provides the user access to the functionality of the MPME 230 and/or MSSS 200 for the purposes described herein.

The exemplary MPME 230 also includes a Notation Retrieval Module (NRM) 250 that is operable to retrieve/receive caregiver-created notation records as exported from the CND 100. In this embodiment, this involves retrieval of documents from the cloud-based data storage 300, although in other embodiments, the storage may be part of the MSSS 200 and/or the records may be received from directly the CNDs 100 rather than be retrieved from the cloud-based storage. The NRM 250 may, and in this embodiment does, store the received caregiver-created notation records as exported from one or more CNDs 100 as Notation Data 224b in the data store 224.

The exemplary MPME 230 further includes a Display Module (DM) 260 that is operable to cause display of appropriate graphical user interface windows and menus/user-selectable menu options via a display device of a computing device (namely, Notes Review Device 400a, 400b, FIG. 1) other than the CND 100 that was operated by the caregiver, such that the caregiver can review that caregiver's own caregiver-created notations as exported from the caregiver's CND 100 on a second computing session (e.g., mobile online, web, personal computer computing platform) different from the mobile computing session of the CND 100, in accordance with the present invention. In some cases, the first computing session may be via a first computing platform (e.g., mobile) and the second computing session may be held via a second computing platform (e.g., web). By way of example, the DM may cause display of appropriate graphical user interface windows and the raw notation records as exported from the CND 100 via a web-based interface of conventional web browser software running on a conventional desktop, laptop or other personal computing device separate from the CND 100, via a website, web portal or other web or Internet-based communications session between the Notes Review Device 400 and the MSSS 200.

The exemplary MPME 230 also includes a Record Creation Module (RCM) 270 that is operable to receive user-input data via the user interface windows and/or interface devices of the Notes Review Device 400, as input to the graphical user interface windows displayed by the DM 260. The RCM 270 works in concert with the DM 260, to cause display of data, receipt of data, and storage of data to allow the raw caregiver-created care notation record as exported by the CND 100 to be reviewed and verified/corrected/augmented/completed via interaction at the Noted Review Device 400, to create the final/definitive patient care documentation record, which is then stored by the RCM 270 as a formal patient record as Record Data 224c in the MSSS 200.

Further, the exemplary MPME 230 also includes an Export Module (EM) 280 that is operable to transmit, synch, and/or export formal patient record data created at the Notes Review Device 400 and/or MSSS 200 to the remote data storage 300 and/or the EMR/EHR system 500, where it can become part of the patient's formal medical/healthcare record. The EM 290 may retrieve stored Export Data 224e, which may indicate how formal record data should be handled, so that the EM 280 can take appropriate action accordingly. For example, the Export Data 224d may provide network address information for transmitting data to the EMR/EHR system 500, and/or to a data store 300. In this way, the EM 290 exports/transmits patient record data (Record Data) to each relevant instance of an EMR/EHR 500 that is a complete/robust care record for each patient, e.g., to comply with industry, regulatory and/or other requirements.

### Caregiver Notation Device Operation

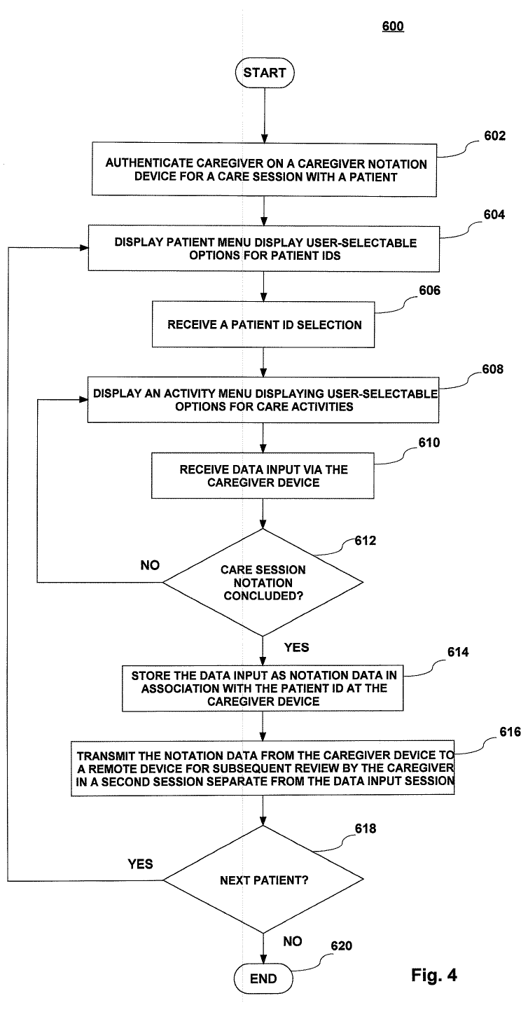

FIG. 4 is a flow diagram 400 illustrating a method of operation of an exemplary Caregiver Notation Device 100 to create informal care documentation notes in accordance with an exemplary embodiment of the present invention. As shown in FIG. 4, the exemplary method begins with authentication of a caregiver on a Caregiver Notation Device 100 for a care session with a patient, as shown at 602. This may be performed by the CAM 140, Display Module 170 and Data Collection Module 180 of the CND 100, working in concert by causing display of a graphical user interface window via the display device 114 of the CND 100 and comparing stored Caregiver Data 124a to login credential information received as user input via an input device of the CND 100.

Next, the exemplary method involves the CND 100 displaying a patient menu displaying user selectable options for patient identifiers, as shown at 604. This may be performed by the PIDM 150, Menu Module 160 and Display Module 170 of the CND 100, working in concert, by causing display of a Patient Identification graphical user interface window via the display device 114 of the CND 100.

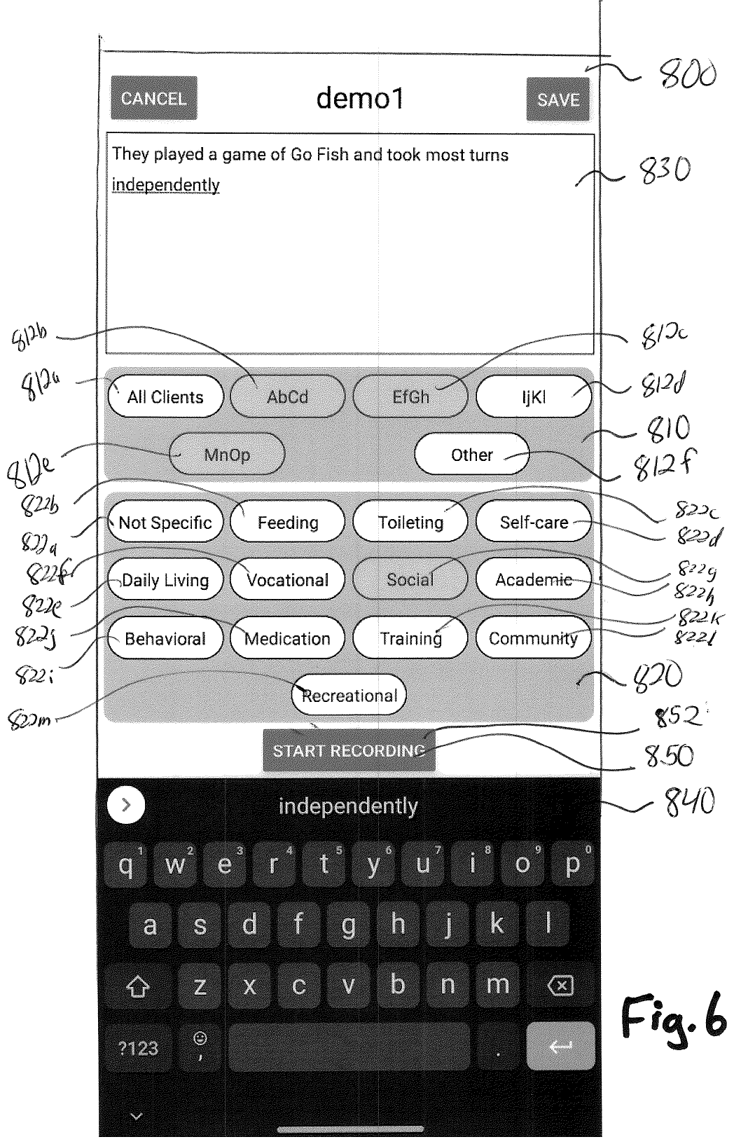

FIG. 6 shows an exemplary graphical user interface window 800 displayed on a display device 114 of an exemplary smartphone-type mobile computing device (CND 100) by the Display Module 170, in accordance with an exemplary embodiment of the present invention. As shown in FIG. 6, the exemplary window 800 includes a displayed exemplary Patient Identification graphical user interface window 810. The window 810 includes a plurality of user-selectable options for patient identifiers, which may be selected to identify the patient to whom the care is being rendered, and in connection with which informal notes are about to be taken in relation to a multi-patient care delivery session.

As will be noted from FIG. 6, the user interface window 800 and Patient Identification graphical user interface window 810 are configured in accordance with the present invention to enables quick, easy, in-the-moment data collection. More particularly, users of the CND 100 can simply click a button to select a patient, list of patient identifiers, range of patient identifiers, etc. at the touch of a single (software-displayed) button 812a, 812b, 812c, 812d, 812e, 812f in the Patient Identification window 810. The Menu Module may determine which options are to be displayed, e.g., as a function of data stored in the Menu Data 124c and/or Patient ID Data 124b, recently-used identifiers, etc.

Next, the exemplary method involves receiving a Patient ID (identifier) selection, as shown at 606. This may be performed by the caregiver selecting (e.g., touching on a touchscreen input device of the CND 100) one of the displayed buttons (e.g., button 812c, which may link to a list of Patient IDs or a next list allowing a caregiver to select a particular Patient ID, etc.), and the Data Collection Module 180 of the CND 100 receiving corresponding user input.

Next, the exemplary method involves the CND 100 displaying an activity menu displaying user selectable options for care activities, as shown at 608. This may be performed by the Menu Module 160 and Display Module 170 of the CND 100, working in concert, by causing display of an Activity Menu graphical user interface window via the display device 114 of the CND 100.

As will be noted from FIG. 6, the user interface window 800 and Activity Menu graphical user interface window 820 are configured in accordance with the present invention to enable quick, easy, in-the-moment data collection. More particularly, users of the CND 100 can simply click a button to select an activity or activity category. at the touch of a single (software-displayed) button 822a, 822b, 822c, 822d, 822e, 822f, 822g, 822h, 822i, 822j, 822k, 822l, 822m in the Activity Menu window 820. The Menu Module may determine which options are to be displayed, e.g., as a function of data stored in the Menu Data 124c, recently-used identifiers, etc.

Next, the exemplary method involves receiving care data input via the CND 100, as shown at 610. This may be performed by the caregiver typing text into a text entry graphical user interface window 830 using a keyboard 840 displayed in the window 800 (e.g., by the system OS and/or the Display Module 170). By way of alternative example, the caregiver may record/enter text into the window 830 using a microphone/transcription function of the graphical user interface window 800 and provided by the OS and/or Display Module 170, which may be initiated by depressing a microphone button (not shown). Alternatively, the caregiver may provide care data input via the CND 100 by making an audio recording using the microphone hardware and other hardware/software of the CND 100, which may be initiated by the caregiver's selection of a Start Recording button 852 displayed in a Record window 850.

In this exemplary method, the method involves next determining whether a particular patient's care session notation has been completed/concluded, as shown at 612. If not, method flow returns to 608, at which point the caregiver may select an additional activity from the Activity Menu 820, provide additional data input, etc., to create a full set of notations documenting the entirety of the patient's care session.

If it is determined that the particular patient's care session notation has been completed/concluded, as shown at 612, then the data input provided by the caregiver in relation to that particular patient's care session notation is stored as Notation Data 124f in the data store 124, in associated with the patient's Patient ID, as shown at 614. This may be performed by the Data Collection Module 180. Further, the DCM 180 may be configured to include additional information in the Notation Data in association with the caregiver-input data. For example, the DCM 180 may be configured to include timestamp for text-based notes voice-base notes and or voice-entered textual notes, by including timestamp (date, time, etc.) information gathered by virtue of the use of conventional smartphone or similar CND's Operating System (OS) software that automatedly timestamps such activities in accordance with aspects of those OS system. Accordingly, this information may be easily and quickly included in the informal notations without a need for the caregiver to provide such information via manual data entry, which eases the recordkeeping burden on the caregiver.

This allows a caregiver to use the caregiver's own smartphone, for example, and to quickly and easily create an informal care notation record documenting care for each of a plurality of patients during a single care session/work shift. The informal records should be quick and easy to complete and need not be a formal and complete care documentation records suitable for inclusion in an EMR, etc., so that the caregiver is not overly burdened during care delivery, deterred from making contemporaneous notes, or distracted from proper care delivery, yet the informal record should be sufficient for future reference and review by the caregiver to create a formal and complete care documentation record suitable for inclusion in an EMR, etc., as discussed below.

Next, the exemplary method involves transmitting the Notation Data from the CND 100 to export the data to a remote device, as shown at 616. The remote device may be a cloud-based data storage 300 and/or the Multi-Session Support System 200. The Notation Data is transmitted to the remote device for further review, augmentation, completion, etc. to form a formal and complete care documentation record suitable for inclusion in an EMR, etc.

Next, the exemplary method involves determine whether the caregiver has a next patient needing care notations during this computing session (care/work shift), as shown at 618. If so, method flow returns to 604 and a caregiver may select a patient ID for a next patient/note from a displayed menu, and the process may be repeated for the next patient/note in a manner similar to that described above, as often as needed during the care session/work shift. If the caregiver does not have a next patient needing care notation during this computing session (care/work shift), then the exemplary method ends, as shown at 618 and 620.

Accordingly, this allows the caregiver to delivery care to multiple patients throughout a day/shift, and to use the CND 100 to create informal care notation records throughout the day/shift that are unique to each patient, and that can be reviewed later, in a next/subsequent computing session, to create a formal and complete care documentation record suitable for inclusion in an EMR, etc.

### Multi-Session Support System Operation

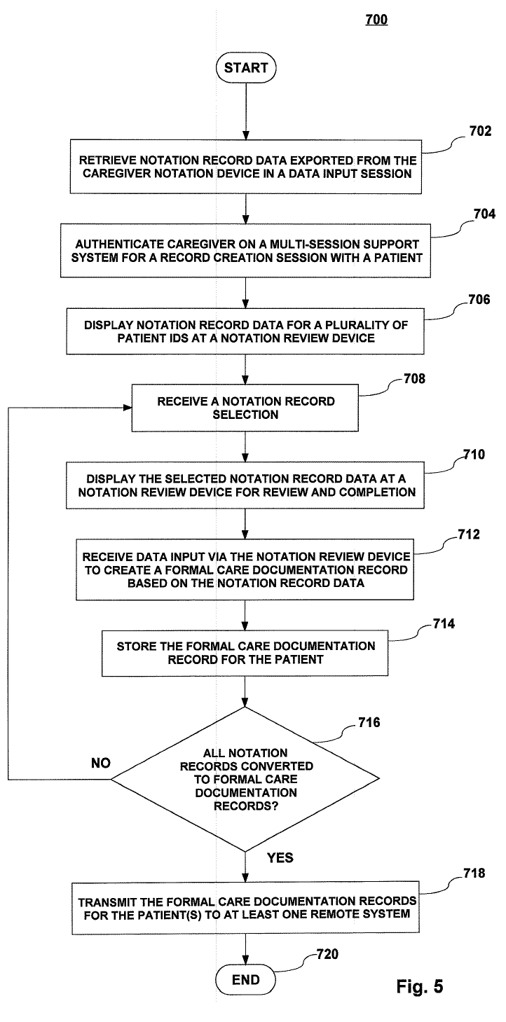

FIG. 5 is a flow diagram 700 illustrating a method of operation of an exemplary Multi-Session Support System (MSSS) 200 to create formal care documentation records in accordance with an exemplary embodiment of the present invention. As shown in FIG. 5, the exemplary method begins with receiving notation record data from at least one CND 100, as shown at 701, which may be transmitted from a CND 100 as a result of a first computing session (notetaking session) at an CND 100. It should be noted that the notation record data may include informal information notation records for care given to one or more patients in a caregiver's single care/work shift. It should be noted that although this example considers primarily export from one CND 100, the MSSS 200 may support and receive data from multiple CNDs operated by multiple caregivers.

At some point after a caregiver's first computing session in which the caregiver used the CND 100 to quickly and easily create informal information notation records documenting care sessions for patients, the caregiver may want to review and correct, augment, complete, etc. each informal information notation record to create a formal care documentation record suitable for inclusion in a patient's medical records, e.g., in an EMR, etc. When the caregiver is ready to do so, the caregiver uses a Notation Review Device 400 (which may be the CND 100 or a desktop/laptop or other computer, which may have a larger display screen and/or otherwise be easier to use for this purpose) to review and edit the information notation records, etc. Accordingly, at such point the caregiver may use the NRD 400 to communicate with the MSSS 200 in a second computing session.

Accordingly, the exemplary method of FIG. 5 next involves the MSSS 200 authenticating a caregiver for a formal care documentation record creation session (second computing session), as shown at 704. This may be performed by the Caregiver Authentication Module 240 and Display Module 270 of the MSSS 200, working in concert by causing display of a graphical user interface window via a display device Notation Review Device 400 and comparing stored Caregiver Data 224a to login credential information received as user input via an input device of the NRD 400.

Next, the exemplary method involves the MSSS 200 displaying notation record data for a plurality of patient identifiers (IDs) at the Notation Review Device 400, as shown at 706. This may be performed by the Notation Retrieval Module 250 and/or the Display Module 260 of the MSSS 200 by network communication with the NRD 400 via the communications network 50, to cause retrieve notation record data (e.g., from the Notation Data 224b and/or cloud-based storage 300) and display of a suitable dashboard graphical user interface window 900 via a display device 114 of the NRD 400.

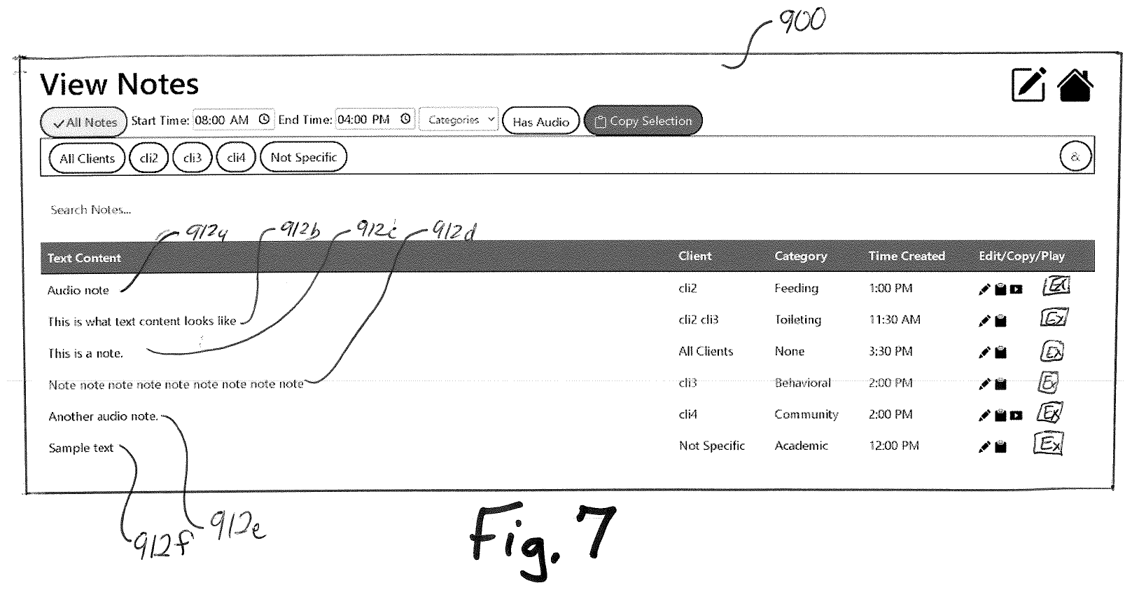

FIG. 7 shows an exemplary graphical user interface dashboard window 900 displayed on a display device of a Notation Review Device 400 by the Display Module 260 of the exemplary Multi-Session Support System 200 in accordance with an exemplary embodiment of the present invention. As shown in FIG. 7, the exemplary dashboard window 900 displays a list of previously-created informal information notation records created by the authenticated caregiver with a variety of filters available for filtering on client, dates, etc. In this exemplary embodiment, the patient identifiers as well as activity categories, and timestamp information are displayed in the list, along with all associated textual, voice recording and/or other information associated with the informal information notation as created by the caregiver at the CND 100.

Next, the exemplary method involves receiving a Notation Record selection, as shown at 708. This may be performed by the caregiver selecting (e.g., clicking or touching on a touchscreen input device of the NRD 4000) one of the displayed options 912a-e. This selection is received and processed by the MSME 230.

Next, the exemplary method involves the MSSS 200 displaying the notation record data for the selected notation record that the NRD 400, for review and editing/completion, as shown at 710. This may be performed by the Notation Retrieval Module 250 and/or Display Module 260 of the MSSS 200, working in concert, by causing display of the notation record in a graphical user interface window via a display device of the NRD 400.

Next, the exemplary method involves receiving care data input via the NRD 400 to create a formal care documentation record suitable for inclusion on the patient's medical record, as shown at 712. This may be performed by the caregiver by reviewing text displayed in the graphical under interface window 900 displayed and the NRD 400 and providing suitable input to correct, augment, supplement, etc. the information notation record data to develop it into a formal and complete care documentation record suitable for inclusion in a medical record of the patient.

This may be performed by the Record Creation Module 270 and/or the Display Module 260 of the MSSS 200, working in concert, by causing display of the suitable graphical user interface windows via a display device of the NRD 400, receiving data input via an input device of the NRD 400 and transmitted over the network 50 to the MSSS 200, etc. The end result of this process is creation of the formal and complete care documentation record suitable for inclusion in a medical record for the patient, based on the caregiver's informal care notations gathered synchronously with the caregiver's delivery of care to the patient, via the CND 100.

Next, the exemplary method involves storing the formal care documentation record for the patient, as shown at 714. This may be performed by the Record Creation Module 270, and result in storage of the record as Record Data 224c in the data store 224 of the MSSS 200, and/or in cloud-based data storage 300.

In this exemplary method, the method involves next determining whether all of the caregiver's notation records have been completed/converted into/included in formal care documentation records, as shown at 716. If not, then method flow returns to 708, at which point the caregiver may select an additional notation record from the dashboard window 900 and provide additional data input, etc., to create a next formal care documentation record, and the process repeats, e.g., until all notation records have been converted to formal care documentation records or until the caregiver wishes to terminate the computing session.

If it is determined that all of the caregiver's notation records have been completed/converted into formal care documentation records (or if the caregiver otherwise wishes to terminate this computing session), then the MSSS 200 transmits the created formal care documentation records and the method ends, as shown at 718 and 710. The created formal care documentation records may include data for one or more patients, and may be transmitted to at least one remote system, such as the appropriate EMR/EHR system 500, for each of the formal care documentation records. By way of example, each formal care documentation record may provide information for determining how/where to transmit the formal care documentation record (e.g., via the network 50) to the particular EMR/EHR system 500, with network address information, etc., which may be included by the Data Management Module 190 of the CND 100 prior to export of the data, although that function may alternatively be performed at the MSSS 200.

This allows a caregiver to use the caregiver's own familiar smartphone/other hardware, for example, and to quickly and easily create an informal care notation record documenting a patient's care session in a first computing session (e.g., on a first computing platform, such as a mobile computing platform), and to subsequently review the notation record and correct/augment/complete it to create a formal care documentation record suitable for inclusion in a medical record of the patient in a second computing session separate from the first computing session (e.g., on a second computing platform, which may be same platform as the first computing session, or different from the platform of the first communication session, such as a web-based computing platform). This allows the caregiver to avoid creation of a formal and complete care documentation record suitable for inclusion in an EMR synchronously with the delivery of care to the patient, so that the caregiver is not overly burdened during care delivery, while still providing for inclusion of a formal and complete care documentation record in the patient's medical record, e.g., in an EMR, etc.

In certain embodiments, the MSSS 200 may be configured with suitable hardware and software for serving as a web server to provides a private secure web portal allowing the user/caregiver to review, sort, filter, and organize the previously-created notation records to facilitate preparation of robust, properly-formatted and complete, end of shift documentation serving as a formal care documentation record.

Notably, the MSSS 200 may contain logic or programming requiring or prompting the user to provide additional information to supplement information contained in the notation record to ensure creation of a complete record (e.g., medical record) for one or more EMR/EHR or other proprietary recordkeeping systems 500, according to the requirements of each system.

Further, the MSSS 200 may contain logic or programming for transmitting the medical record information via a communications network 60 to an appropriate EMR/EHR or other proprietary recordkeeping systems 500 (e.g., one of the health systems providing the services to the patient/person).

Still further, the Notation Review Device 400 may contain logic or programming for formatting the medical record information into a suitable proprietary or common file format to ensure creation of a usable record (e.g., medical record) that is compatible with one or more EMR/EHR or other proprietary recordkeeping systems 500.

The MSSS 200 may provide a dashboard window including features allowing caregivers to track tasks and share notes with one another, and to make the system HIPAA compliant. For example, in addition to direct support/caregiving tasks, DSPs also have tasks associated with maintaining a safe environment, such as cleaning, shopping, checking fire alarm batteries, etc. Many tasks happen daily, others at less frequent cadences. These tasks can be divided up among the DSPs who are working in the same location during the same shift. In such an embodiment, the dashboard window may display those facility/work location tasks and who is assigned to complete each one and the task completion status.

Using this multi-session (e.g., phone-to-web) system, the caregiver can then also use the relevant employer's selected proprietary record keeping system, and then, within the OS of a personal computing device or similar device, copy and paste notes taken during the day into each appropriate medical record of the employer's selected proprietary record keeping system, to allow for efficient and effective recordkeeping. This approach provides an overall lessening of the burden of post-shift documentation of home health care visits.

It should be noted that in alternative embodiments of the present invention, the MSSS 200 may simply allow records to be selected and displayed (and possibly edited) at the NRD 400 (e.g., as described at 708 and 710, and possibly 712) without the subsequent editing, formal record creation/storage, transmission to an EMR, etc. by the MSSS 200 as described in steps 712-718. In such embodiments, the MSSS 200 may cause only display (or possibly editing) at the NRD 400, and allow for selection of the information notation record content for subsequent copying into another graphical user interface window of another software application, which will then be used to edit/complete a formal care documentation record, store the documentation record, and/or upload. For example, the copy-and-paste function of the operating system software of the NRD 400 may be used to copy the information notation records caused to be displayed by the MSSS 200 at the NRD 200, and then to paste that content into a draft document of a conventional word processing software's graphical user interface window displayed at the NRD 200, which may then be used to edit/augment, etc. and create the formal care documentation, or to paste that content into a graphical user interface window of proprietary software of the caregiver's employer that is displayed at the NRD 400 (e.g., as part of conventional proprietary recordkeeping software, or EMR/EHR software or associated browser-displayed or other graphical user interface windows that are outside the scope of the present invention).

Also provided are computer program products for implementing the method described herein. Each computer program product comprises a non-transitory computer-readable medium storing executable instructions that, when executed by a processor, cause a computerized system to perform a method described herein.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A computerized caregiver notation device configured to record care information notations for review in a separate computing session, the caregiver notation device comprising:
a memory comprising a non-transitory data processor-readable medium;
a display device;
a data processor operatively connected to said memory and display device;
a user interface device operatively connected to said data processor;
user interface management instructions embodied in data processor-executable code stored in said memory, said user interface management instructions being executable by the data processor to provide a user interface management engine configured to:
display, via said display device, at least one graphical user interface window operable to authenticate a caregiver for a care session with at least one patient;
display, via said display device, a patient menu displaying user selectable options for patient identifiers;
display, via said display device, an activity menu displaying user selectable options for care activities;
receive, via said user interface device, user input comprising at least one patient identifier selected from the patient menu and at least one care activity selected from the activity menu;
store, in said memory, said user input as an informal care information notation record; and
transmit, via a communications network, data identifying said informal care information notation record to a remote system.

2. The computerized caregiver notation device of claim 1, wherein said user interface management instructions configured to display, via the display device, at least one graphical user interface window operable to authenticate a caregiver for a care session with at least one patient comprise instructions to configure the user interface management engine to compare stored data of an authorized caregiver to login credential information received as user input provided via said at least one graphical user interface window via said user interface device.

3. The computerized caregiver notation device of claim 1, wherein said user interface management instructions configured to display, via said display device, an activity displaying user selectable options for care activities further comprise instructions to configure the user interface management engine to receive care data input selected from the activity menu and provided as user input via said at least one graphical user interface window via said user interface device.

4. The computerized caregiver notation device of claim 1, wherein said user interface management instructions further comprise instructions to configure the user interface management engine to display, via said display device, a text entry graphical user interface window operable to receive care data input.

5. The computerized caregiver notation device of claim 4, wherein said user interface management instructions to display, via said display device, the text entry graphical user interface window operable to receive care data input further comprise instructions to configure the user interface management engine to receive care data input provided as user input via said at least one graphical user interface window via said user interface device.

6. The computerized caregiver notation device of claim 1, wherein said user interface management instructions further comprise instructions to configure the user interface management engine to add timestamp data to said informal care information notation record.

7. The computerized caregiver notation device of claim 1, wherein said user interface management instructions to transmit data identifying said informal care information notation record to the remote system comprise instructions to configure the user interface management engine to transmit said to a computerized notation review device for at least one of review and editing during a subsequent computing session.

8. A computerized multi-session support system configured to permit review in a second computing session of informal care information notations recorded in a separate first computing session, the multi-session support system comprising:
a memory comprising a non-transitory data processor-readable medium;
a data processor operatively connected to said memory;
multi-session management instructions embodied in data processor-executable code stored in said memory, said multi-session management instructions being executable by the data processor to provide a multi-session management engine configured to:
receive, via a communications network, data comprising at least one informal care information notation record comprising data gathered from a caregiver via a caregiver computing device during a first computing session;
transmit, via the communications network to a computerized notation review device comprising a display device, data to cause display, via the display device, at least one graphical user interface window operable to receive data for authenticating the caregiver;
receive, via the communications network from the computerized notation review device, data authenticating the caregiver; and
transmit, via the communications network to the computerized notation review device, data to cause display, via the display device, said data comprising said at least one informal care information notation record to permit said caregiver to review said comprising said at least one informal care information notation record in the second computing session via the computerized notation review device.

9. The computerized multi-session support system of claim 8, wherein said user interface management instructions further comprise instructions to configure the multi-session management engine to receive, via the communications network from the computerized notation review device, data augmenting the informal care information notation record to create a corresponding formal care documentation record.

10. The computerized multi-session support system of claim 9, wherein said user interface management instructions further comprise instructions to configure the multi-session management engine to transmit, via the communications network to a cloud storage device, the corresponding formal care documentation record.

11. The computerized multi-session support system of claim 9, wherein said user interface management instructions further comprise instructions to configure the multi-session management engine to transmit, via the communications network to an electronic medical records system, the corresponding formal care documentation record.

12. The computerized multi-session support system of claim 9, wherein said user interface management instructions further comprise instructions to configure the multi-session management engine to transmit, via the communications network to the computerized notation review device, data to cause display, via the display device, of a graphical user interface window prompting the caregiver to provide additional information as input to the notation review device to create the corresponding formal care documentation record by augmenting the informal care information notation record.

13. The computerized multi-session support system of claim 9, wherein said user interface management instructions further comprise instructions to configure the multi-session management engine to format the formal care documentation record into a format compatible with an electronic medical records system.

14. The computerized multi-session support system of claim 8, wherein said user interface management instructions further comprise instructions to configure the multi-session management engine to provide the second computing session on a computing platform distinct from a respective computing platform of the first computing session.

15. The computerized multi-session support system of claim 8, wherein said user interface management instructions further comprise instructions to configure the multi-session management engine to provide the second computing session on a web computing platform.

16. A computer program product comprising a non-transitory computer-readable medium storing executable instructions that, when executed by a processor, cause a computerized system to perform a method comprising:
displaying, via a display device, at least one graphical user interface window operable to authenticate a caregiver for a care session with at least one patient;
displaying, via the display device, a patient menu displaying user selectable options for patient identifiers;
displaying, via the display device, an activity menu displaying user selectable options for care activities;
receiving via the user interface device, user input comprising at least one patient identifier selected from the patient menu and at least one care activity selected from the activity menu;
storing, in a memory, said user input as an informal care information notation record; and
transmitting, via a communications network, data identifying said informal care information notation record to a remote system.

17. The computer program product of claim 16, further comprising executable instructions that, when executed by a processor, cause a computerized system to perform a method comprising receiving care data input selected from the activity menu and provided as user input via said at least one graphical user interface window via a user interface device.

18. The computer program product of claim 16, further comprising executable instructions that, when executed by a processor, cause a computerized system to perform a method comprising displaying, via the display device, a text entry graphical user interface window operable to receive care data input.

19. The computer program product of claim 16, further comprising executable instructions that, when executed by a processor, cause a computerized system to perform a method comprising receiving care data input provided as user input via said at least one graphical user interface window via said user interface device.

20. The computer program product of claim 16, further comprising executable instructions that, when executed by a processor, cause a computerized system to perform a method comprising adding timestamp data to said informal care information notation record.

21. The computer program product of claim 16, further comprising executable instructions that, when executed by a processor, cause a computerized system to perform a method comprising transmitting said to a computerized notation review device for at least one of review and editing during a subsequent computing session.

22. A computer program product comprising a non-transitory computer-readable medium storing executable instructions that, when executed by a processor, cause a computerized system to perform a method comprising:
receiving, via a communications network, data comprising at least one informal care information notation record comprising data gathered from a caregiver via a caregiver computing device during a first computing session;
transmitting, via the communications network to a computerized notation review device comprising a display device, data to cause display, via the display device, at least one graphical user interface window operable to receive data for authenticating the caregiver;
receiving, via the communications network from the computerized notation review device, data authenticating the caregiver; and
transmitting, via the communications network to the computerized notation review device, data to cause display, via the display device, said data comprising said at least one informal care information notation record to permit said caregiver to review said comprising said at least one informal care information notation record in a second computing session via the computerized notation review device.

23. The computer program product of claim 22, further comprising executable instructions that, when executed by a processor, cause a computerized system to perform a method comprising receiving, via the communications network from the computerized notation review device, data augmenting the informal care information notation record to create a corresponding formal care documentation record.

24. The computer program product of claim 23, further comprising executable instructions that, when executed by a processor, cause a computerized system to perform a method comprising transmitting, via the communications network to a cloud storage device the corresponding formal care documentation record.

25. The computer program product of claim 23, further comprising executable instructions that, when executed by a processor, cause a computerized system to perform a method comprising transmitting, via the communications network to an electronic medical records system the corresponding formal care documentation record.

26. The computer program product of claim 22, further comprising executable instructions that, when executed by a processor, cause a computerized system to perform a method comprising transmitting, via the communications network to the computerized notation review device, data to cause display, via the display device, of a graphical user interface window prompting the caregiver to provide additional information as input to the notation review device to create a corresponding formal care documentation record by augmenting the informal care information notation record.

27. The computer program product of claim 23, further comprising executable instructions that, when executed by a processor, cause a computerized system to perform a method comprising formatting the formal care documentation record into a format compatible with an electronic medical records system.
